(19) United States
(12) Patent Application Publication
(10) Pub. No.: US 2024/0424017 A1
(43) Pub. Date: Dec. 26, 2024

(54) NANOPARTICLE (NP)-ENHANCED EXPRESSION OF AQUAPORIN-4 CHANNELS AND WATER TRANSPORT IN HUMAN ASTROCYTES

(71) Applicant: The Government of the United States of America, as represented by the Secretary of the Navy, Arlington, VA (US)

(72) Inventors: Katherine Rogers, Silver Spring, MD (US); Okhil K. Nag, Brandywine, MD (US); Michael H. Stewart, Springfield, VA (US); Eunkeu Oh, Alexandria, VA (US); Kimihiro Susumu, Alexandria, VA (US); James B. Delehanty, Washington, DC (US)

(73) Assignee: The Government of the United States of America, as represented by the Secretary of the Navy, Arlington, VA (US)

(21) Appl. No.: 18/750,592

(22) Filed: Jun. 21, 2024

Related U.S. Application Data
(60) Provisional application No. 63/522,930, filed on Jun. 23, 2023.

(51) Int. Cl. A61K 33/30; A61K 33/24; A61K 33/242; A61K 47/54; A61K 47/64

(52) U.S. Cl. CPC A61K 33/30; A61K 33/24; A61K 33/242; A61K 47/542; A61K 47/64

(57) ABSTRACT

A method of inducing expression of aquaporin-4 by providing a bioconjugate having a quantum dot bound to human erythropoietin and contacting human astrocytes with the bioconjugate, which induces expression of aquaporin-4. A composition having a bioconjugate having a quantum dot bound to human erythropoietin. A method of providing a quantum dot and forming a bioconjugate by depositing human erythropoietin onto the surface of the quantum dot.

### REFERENCE TO RELATED APPLICATIONS

This application claims the benefit of U.S. Provisional Application No. 63/522,930, filed on Jun. 23, 2023. The provisional application and all other publications and patent documents referred to throughout this nonprovisional application are incorporated herein by reference.

### TECHNICAL FIELD

The present disclosure is generally related to carbon/polymer materials for battery discharge.

### DESCRIPTION OF THE RELATED ART

The glymphatic system in the brain mediates the removal of extracellular proteins, wastes, and solutes. Because the brain does not have a lymphatic circulation, wastes must be cleared by an alternative mechanism. This is achieved by the movement of cerebralspinal fluid (CSF) through the interstitial space by the water transport action of support cells called astrocytes (see ref. 3). The water-pumping action is carried out by water-conducting membrane channel proteins called aquaporins (specifically aquaporin-4, AQPN-4). This system has been shown to function nearly exclusively during sleep, providing the brain with the necessary “flushing” that is required to maintain optimal cognitive function. This poses a considerable challenge for those in situations causing sleep debt and the concomitant degradation of cognitive function.

There has been considerable interest in the development of drugs that can target AQPN-4 function, but to date limited progress has been made. The controlled induction of AQPN-4 expression is a new paradigm with potential impact for the treatment of traumatic brain injury, stroke, and Alzheimer's and Parkinson's diseases.

A need exists for techniques to induce AQPN-4 expression in astrocytes.

### SUMMARY OF THE INVENTION

Described herein is a technique for the controlled, induced upregulation of expression of AQPN-4 channel proteins in astrocyte cells by incubation of the cells with erythropoietin (EPO) in the form of an NP-EPO conjugate. A nanoparticle (NP), namely a fluorescent semiconductor quantum (QD) is used as a luminescent, trackable scaffold to present EPO to the astrocyte plasma membrane in a multivalent configuration. This effectively simultaneously ligates multiple EPO receptors, resulting in the activation of the intracellular signaling pathway that instructs the cell to synthesize and display AQPN-4 channel proteins on the plasma membrane. The multivalent display of EPO on the QD surface results in a ˜1.8-fold greater increase in the expression levels of AQPN-4 channels compared to free EPO at the same concentration, with a 2-fold increase in the water efflux rate from astrocytes.

In one embodiment, a method of inducing expression of aquaporin-4 comprises providing a bioconjugate comprising a quantum dot bound to human erythropoietin and contacting human astrocytes with the bioconjugate, thereby inducing expression of aquaporin-4.

In another embodiment, a composition comprises a bioconjugate comprising a quantum dot bound to human erythropoietin.

In another embodiment, a method comprises providing a quantum dot and forming a bioconjugate by depositing human erythropoietin onto the surface of the quantum dot.

### BRIEF DESCRIPTION OF DRAWINGS

FIGS. 1A and 1B schematically depict an EPO-QD (erythropoietin—quantum dot) bioconjugate system (note that these figures are not to scale). FIG. 1A shows the bioconjugate system comprising CdSe-CdS/ZnS core-shell QDs capped with the zwitterionic ligand CL4. The shell comprises an inner CdS shell and an outer ZnS shell. Erythropoietin (EPO) bearing a C-terminal polyhistidine domain (EPO-his, in red) is assembled to the QD surface via metal affinity interaction between the his domain and ZnS shell. (B) The multivalent display of EPO-his enables ligation of multiple EPO receptors (EPOR) for enhanced activation of the Janus kinase/signal transducer and activator of transcription (JAK/STAT) signaling pathway resulting in the augmented synthesis and localization of aquaporin-4 (AQPN-4) to the plasma membrane.

FIGS. 2A and 2B relate to the physical characterization of EPO-QD bioconjugates. FIG. 2A shows results from gel electrophoresis analysis of QDs and EPO-QD conjugates. The EPO/QD ratio is shown along with the negative (cathode) and positive (anode) electrodes. The arrow indicates the location of the sample loading wells. FIG. 2B provides dynamic light scattering (for determination of Dh) and zeta potential (surface charge) analysis for QDs and EPO-QD conjugates.

FIGS. 3A-3C show quantification of the binding of EPO-QD to EPO receptor (EPOR) on human astrocytes (HA). Micrographs in FIG. 3A show the resulting fluorescence when 10 nM QDs alone or 10 nM QDs assembled with the EPO concentrations indicated were incubated with HA for 24 h followed by washing and imaging. 20, 50, and 100 nM EPO correspond to EPO/QD ratios of 2, 5, and 10, respectively. FIG. 3B is a quantification of fluorescence images showing the increase in fluorescence signal of the membrane-bound QDs that reaches a maximum at a ratio of 5 EPO/QD. There is no statistical significance between 5 and 10 EPO/QD. FIG. 3C provides a time-resolved quantification of EPO-QD (at 5 EPO/QD) binding to astrocytes. The EPO-QD conjugates were incubated on the cells for the times indicated. In panels B and C, the data correspond to analysis of at least 100 cells across two independent experiments and * indicates p<0.01.

FIGS. 4A-4C depict the induction of AQPN-4 expression by free EPO and EPO-QD. FIG. 4A shows micrographs after EPO-QD appended with EPO protein at 20, 50, or 100 nM (corresponding to 2, 5, or 10 copies/QD) (top row) or free EPO (bottom row) were incubated on HA for 24 h and then cells were probed with antibodies to quantify AQPN-4 expression. QDs were used at 10 nM when present. FIG. 4B provides a quantification of AQPN-4 expression showing that EPO-QD induces a greater level of AQPN-4 expression compared to free EPO at the same concentration. 5 EPO/QD provided the best results such that AQPN-4 induction was increased by 1.8-fold. * indicates p<0.05; ** indicates p<0.001. The data correspond to analysis of at least 100 cells across two independent experiments. FIG. 4C shows that the level of AQPN-4 expression tracks directly with the level of EPO-QD binding to the plasma membrane.

FIGS. 5A-5C provide a quantification of water transport in EPO-treated HA using calcein AM assay. A calcein AM quenching assay was used to visibly track and quantify the rate of water efflux in HA treated with EPO-QD or free EPO. Calcein AM fluorescence quenches with increasing dye concentration. When water effluxes out of the cell, calcein AM concentration increases, resulting in its quenched fluorescence. FIG. 5A contains micrographs of time-resolved calcein AM quenching. After incubation with 50 nM EPO-QD (10 nM QD/5 EPO per QD) for 24 h, HA were loaded with calcein AM. Starting in physiological salt conditions (300 mOsmol, isotonic) at T0 sec (high calcein fluorescence), the extracellular solution was made hypertonic (˜500 mOsmol) at T20 sec by addition of NaCl (black arrow). Calcein AM fluorescence begins to quench at T20 sec, indicative of water efflux. The white arrows highlight two cells in each frame for ease of tracking fluorescence change. FIG. 5B is a plot of the comparative calcein AM quenching extracted from the images in panel A. Each data trace corresponds to the average of 60 cells and is representative of four independent experiments. FIG. 5C plots the calcein AM fluorescence response over the time period T20 sec-T30 sec (from the traces in panel B) are fit to the exponential identity y=−Ce^{(-kt)} where k is proportional to the membrane water permeability or water transport rate. The curve fit values (R2) are: 50 nM EPO-QD, 0.94; 50 nM EPO, 0.96; QD only, 0.98; and no EPO/no QD, 0.98.

FIG. 6 shows quantification of relative water transport rates in EPO-treated HA. Plotted are the water transport rates determined from the curve-fit of the quenching plots in FIG. 5C. *** indicates p<0.001. The values of k (the water permeability rate constant) were found to be as follows: 50 nM EPO-QD, k=0.103; 50 nM EPO, k=0.055; 10 nM QD control, k=0.044; untreated cell control (no EPO/no QD), k=0.022.

FIG. 7 contains data on a test of the possible cytotoxicity of EPO and EPO-QD bioconjugates. HA cell monolayers were incubated for up to 48 hr with the materials as indicated and then a live/dead cell analysis (calcein/ethidium homodimer 1) was performed. The data show greater than 96% cell viability for all treatment conditions and the control (no EPO/no QD).

### DETAILED DESCRIPTION

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein, the singular forms “a” “an,” and “the” do not preclude plural referents, unless the content clearly dictates otherwise.

As used herein, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

### Overview

As described herein, the presentation of the growth factor EPO to the astrocyte plasma membrane in a multivalent form on the surface of a nanoparticle (NP) acts to stimulate greater synthesis and membrane-display of AQPN-4 compared to free, monomeric EPO, resulting in increased water efflux from astrocytes. EPO has been shown to induce the expression of AQPN-4 water channels in vivo via its binding to the EPO receptor (EPOR) and activation of the JAK/STAT intracellular signaling pathway (see ref. 4).

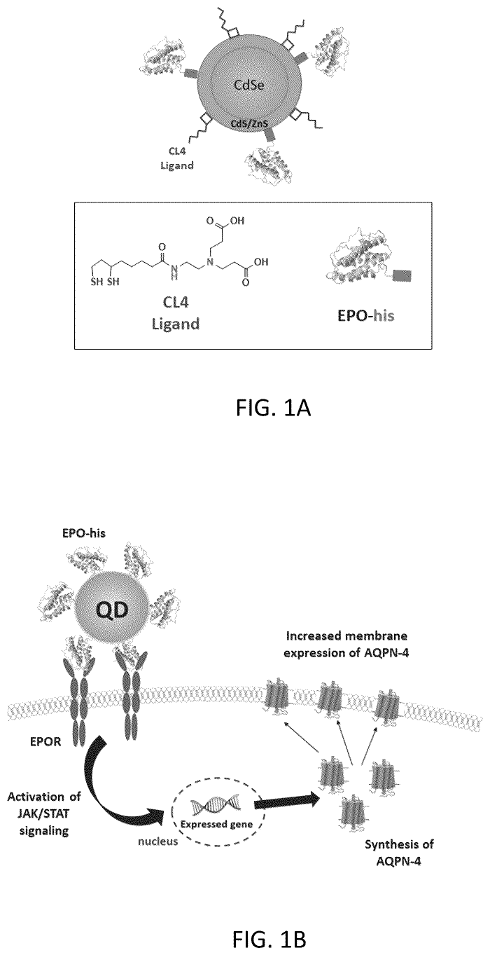

A fluorescent semiconductor quantum dot (QD) NP is used as a luminescent, trackable scaffold to present EPO to the astrocyte plasma membrane in a multivalent configuration. This effectively simultaneously ligates multiple EPO receptors, resulting in the activation of the intracellular signaling pathway that instructs the cell to synthesize and display AQPN-4 channel proteins on the plasma membrane. As described below, the multivalent display of EPO on the QD surface results in a ˜1.8-fold greater increase in the expression levels of AQPN-4 channels compared to free EPO at the same concentration. This is shown schematically in FIGS. 1A and 1B. This, in turn, is coupled with a 2-fold increase in the water efflux rate from astrocytes.

### EXAMPLES

FIG. 1A schematically depicts the self-assembly of EPO bearing a C-terminal six-histidine tract (EPO-his) to the surface of CdSe-CdS/ZnS core-shell QDs. These QDs (˜5 nm in diameter) exhibit excellent crystallinity as shown by TEM with emission centered at 540 nm. The histidine ‘tail’ on the EPO binds to the ZnS shell on the QD surface via metal affinity coordination and allows for the ratiometric assembly of EPO proteins to the QD. These experiments used 540 nm-emitting QDs (˜5 nm diameter) whose surface was capped with the solubilizing ligand CL4. This ligand is short (˜1.5 nm in length) and it allows the histidine tail to access the QD surface. It also imparts a net negative charge to the QD surface. The CL4 ligand is described in U.S. Pat. No. 8,512,755.

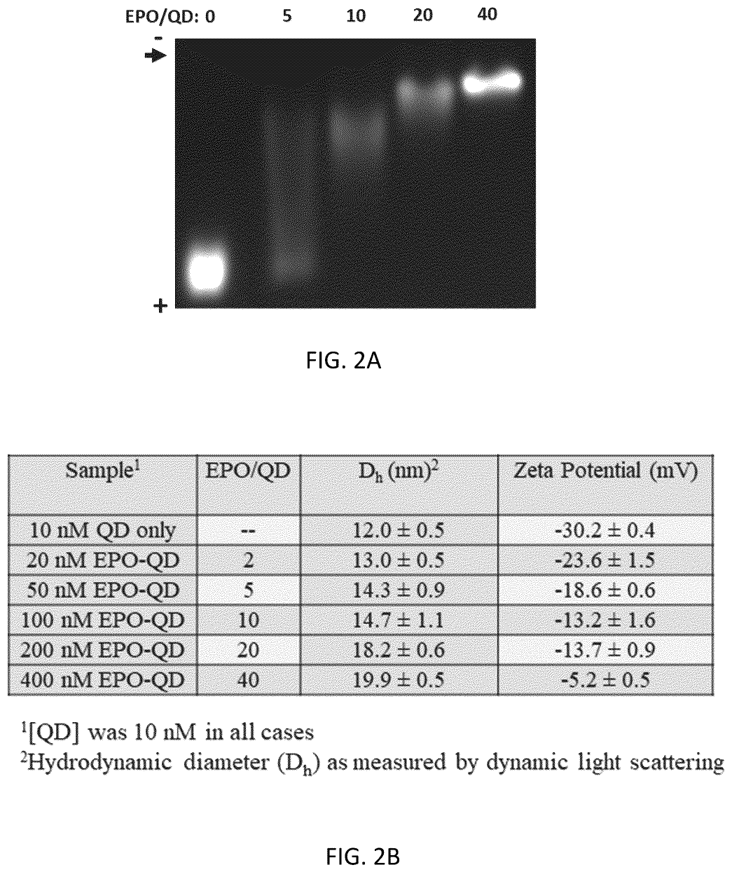

The assembly of EPO-his to the QD surface was confirmed by gel electrophoresis, dynamic light scattering (DLS), and zeta potential measurements. FIG. 2A shows that when assembled with increasing ratios of EPO-his, the gel migration of the negatively-charged QDs towards the anode (positive post) is slowed. This is consistent with both the shielding of the QD surface charge and the formation of larger conjugates when QDs are assembled with increasing ratios of EPO-his. Based on the gel migration, a ratio of 20 EPO-his per QD resulted in maximum QD surface loading as addition of EPO-his beyond this ratio did not substantially further retard QD movement. This agrees well with the predicted QD surface packing density based on the available QD surface area and the size/molecular weight (30 kDa) of the EPO where ˜20 EPO is predicted to saturate the QD surface. DLS analysis and zeta potential showed a consistent increase in hydrodynamic diameter (Dh) and a decrease in overall negative QD surface charge with increasing addition of EPO-his, respectively (FIG. 2B). Cumulatively, these results confirm the controlled, ratiometric assembly of EPO-his to the QD surface.

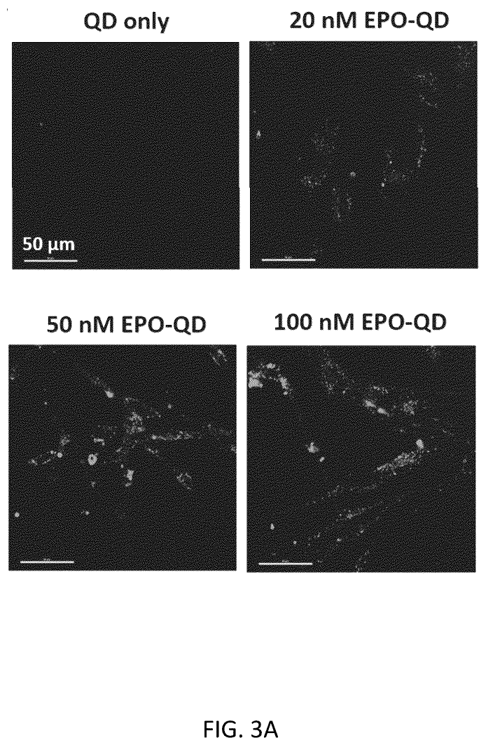

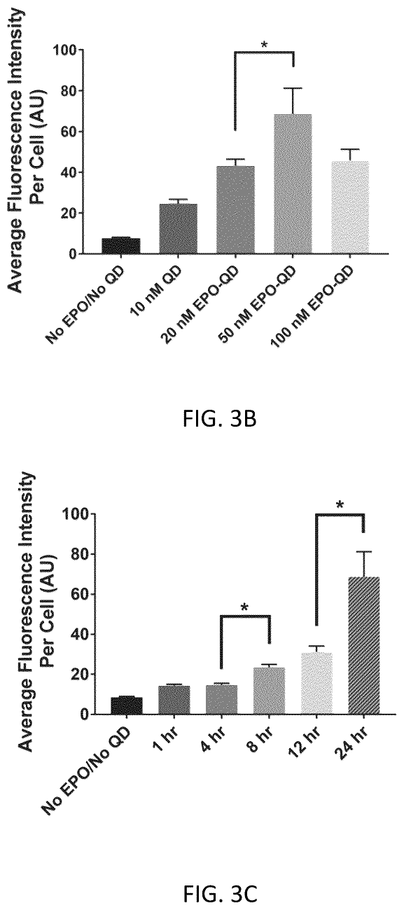

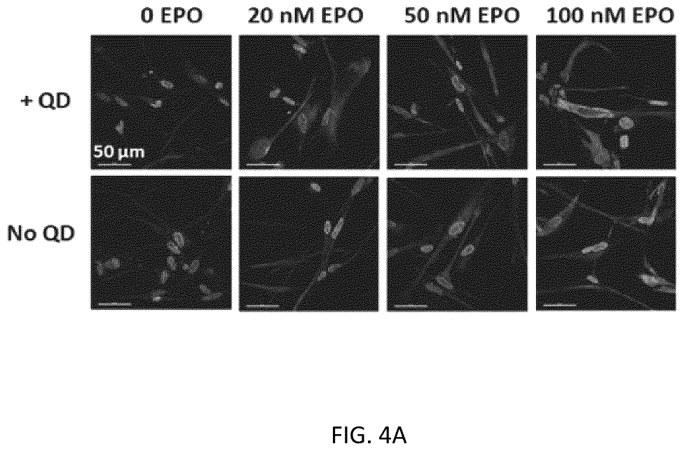

The EQO-QD conjugates were then examined for binding to the cell surface of human astrocytes (HA) by virtue of their binding to the EPO receptor (EPOR), which is natively expressed by these cells. HA were incubated for 24 h with 10 nM QDs assembled with increasing amounts of EPO-his (20, 50, and 100 nM corresponding to EPO/QD ratios of 2, 5, and 10, respectively) then washed to remove unbound QD. FIG. 3A shows the resulting fluorescence micrographs with corresponding quantified data presented in FIG. 3B. Interestingly, the data show that the fluorescence intensity on the cell surface increases up to a ratio of 5 EPO/QD where the degree of QD binding is maximal. Above this ratio, no further increase in QD binding is observed. This observation is consistent with a “steric hindrance” effect wherein at a ratio of 5 EPO/QD on a 5 nm diameter QD, the EPO is presented to the cell's plasma membrane in such a way that maximal binding of receptors is achieved; increasing the number of EPO/QD beyond that does not increase the amount of QDs bound to the cell. It is expected that this effect could be overcome by using a larger QD with greater surface area combined with increasing the separation distance between the EPO and the QD surface to alleviate the steric hindrance effect. Time-resolved imaging over a 24 h time period where HA were incubated with 10 nM QD (5 EPO/QD) showed a steady increase in the amount of cellular QD labeling (FIG. 3C). Further, the EPO-QD conjugates were largely colocalized with an AlexaFluor546-transferrin endocytosis marker (data not shown). This is consistent with the known EPO-induced endocytosis of EPOR which is regulated by JAK kinase activity. Control, competition binding experiments where cells were co- incubated with a 50 nM EPO/10 nM QD and a five-fold molar excess of free EPO showed significant inhibition of EPO-QD binding to HA cells, demonstrating the specificity of interaction of EPO-QD with the EPOR (data not shown).

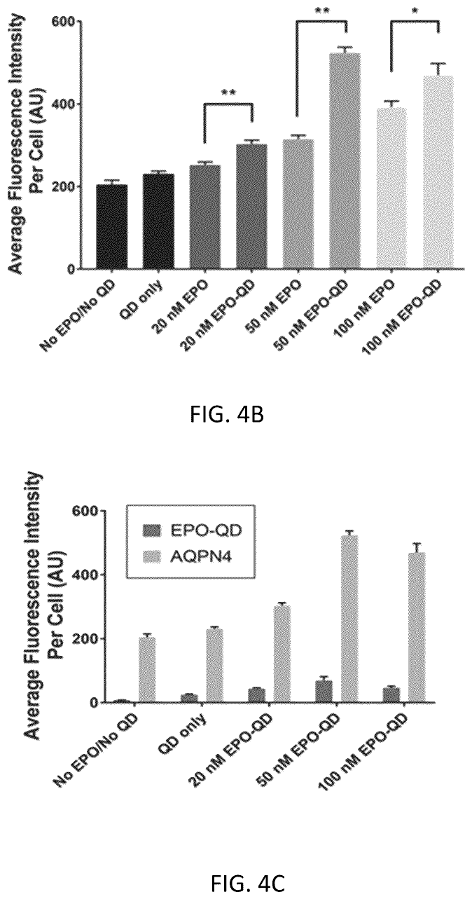

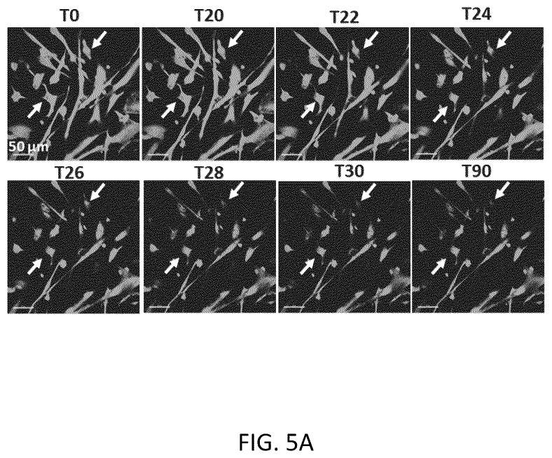

Having demonstrated the quantitative nature of EPO-QD binding to the astrocyte cell surface, the EPO-QD-mediated induction of expression of cell-surface AQPN-4 channel proteins was examined. To achieve this, astrocytes were incubated for 24 h with EPO-QD (in particular, with 10 nM QD having 2, 5, or 10 EPO assembled to the QD surface) or free EPO at the equivalent concentrations (20, 50, or 100 nM). The 24 h incubation period was chosen to ensure ligation of the EPO receptor and activation of the intracellular JAK/STAT signaling pathway that instructs the cell to synthesize AQPN-4 protein and translocate it to the plasma membrane. After the incubation period, cells were washed, fixed and permeabilized, and probed with an anti-AQPN-4 primary antibody. This was followed by staining with a fluorescently-labeled (Texas Red) secondary antibody to visualize the level of AQPN-4 channel expression. The fluorescence micrographs in FIG. 4A show the resulting staining when astrocytes were incubated with EPO-QD (top row) or free EPO (bottom row). The quantified results are shown in FIG. 4B. It was apparent that the presentation of EPO to the astrocyte plasma membrane in a multivalent form on the surface of the QD more efficiently induced AQPN-4 expression compared to free EPO at the same concentration. At all three EPO/QD ratios, there was a statistically significant increase in AQPN-4 expression induced by EPO-QD over free EPO alone at the same concentration. A ratio of 5 EPO/QD (50 nM EPO/10 nM QD) was the best case wherein AQPN-4 expression was induced by -1.8 fold (80% increase). Further, the level of AQPN-4 expression induction tracks directly with the level of EPO-QD binding to the plasma membrane (FIG. 4C).

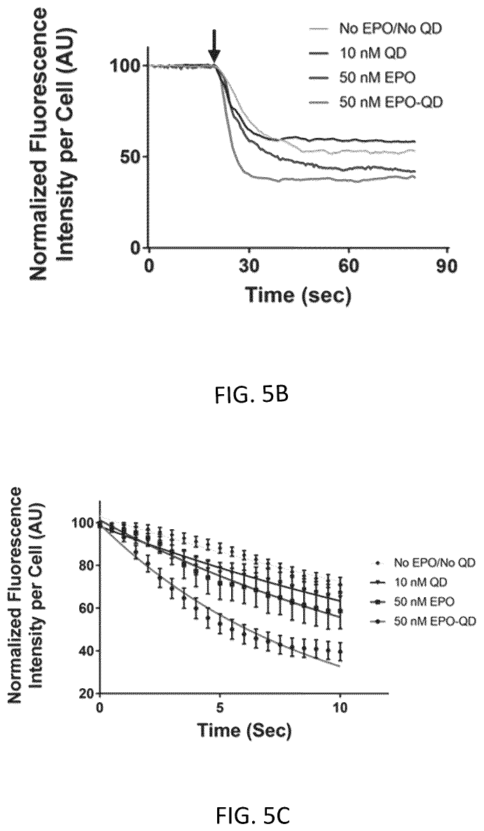

Preferably, the increased expression of AQPN-4 induced by EPO-QD results in increased water transport in treated HA cells. Accordingly, tests were done using fluorescence- based water transport assays to determine the relative rates of water efflux from astrocytes treated with EPO-QD or free EPO. For this, a calcein-AM quenching assay was used. Intracellularly, the quenching of calcein-AM fluorescence tracks inversely with its concentration. At physiological salt (300 mOsmol) calcein fluorescence is high. When the extracellular media is made hypertonic (500 mOsmol) by the addition of NaCl, water effluxes from the cell, causing the calcein concentration to increase and its fluorescence to quench. FIG. 5A shows fluorescence micrographs of astrocytes that were incubated with 50 nM EPO-QD (10 nM QD; 5 EPO/QD) for 24 hours and subsequently loaded with calcein-AM. At T20 sec the extracellular media was made hypertonic, inducing water to efflux from the cells which occurred rapidly in the 10 sec window T20 sec-T30 sec and plateaued through T90 sec. FIG. 5B shows the time-resolved comparative fluorescence traces of astrocytes under various treatment conditions. Cells treated with 50 nM EPO-QD exhibited the steepest calcein-AM fluorescence decrease in that time window that was greater than that of cells treated with 50 nM free EPO, 10 nM QDs alone, or untreated control cells. This is direct evidence of the faster rate of water efflux in these cells. FIG. 5C shows plots of the comparative rates of calcein-AM fluorescence decrease over the 10 sec time window from T20 sec to T30 sec. It is clear from the data that cells treated with 50 nM EPO-QD exhibited a significantly greater rate of calcein-AM quenching compared to cells treated with free EPO, indicating a faster rate of water efflux. The curves are fit to the exponential identity:

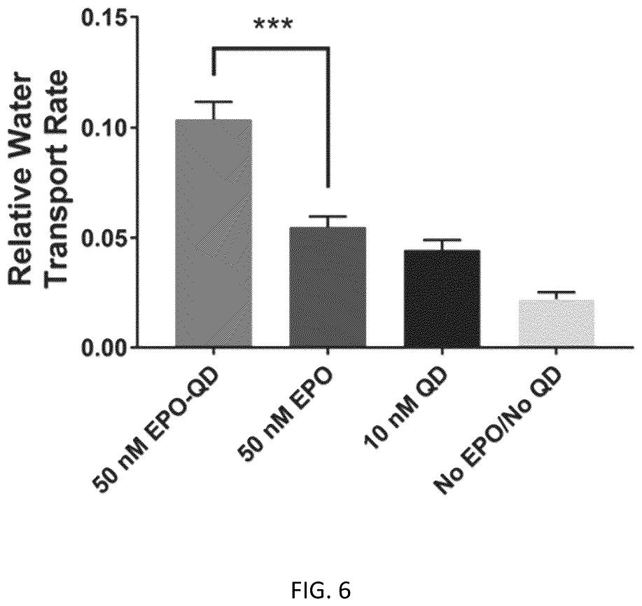

y=1−Ce^{(-kt)}

where k is proportional to the water membrane water permeability or water transport rate.6 FIG. 6 shows the calculated relative water transport rates for astrocytes treated with EPO-QD, free EPO, QDs alone, or control cells (derived from the curve fitting in FIG. 5C). It was apparent that astrocytes treated with 50 nM EPO-QD exhibited a 2-fold greater water efflux rate compared to cells treated with 50 nM free EPO, as can be seen in FIG. 6.

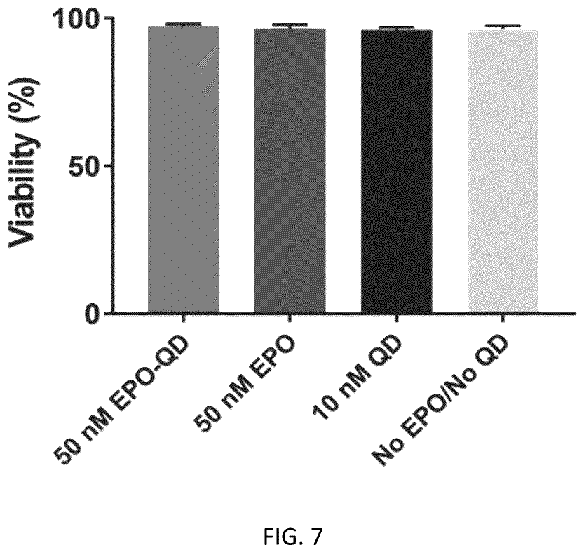

The effect of the incubation of HA with EPO-QD (or free EPO) on HA cellular proliferation was examined under the conditions required to induce the increased expression of AQPN-4. Controls again included HA cells incubated with 10 nM QDs alone or untreated cells (medium only) (FIG. 7). No statistical difference in cell viability were seen between cells treated with the EPO-QD conjugates, EPO alone, or QDs alone compared to untreated control cells. These results demonstrate not only the negligible impact on HA cellular health in the context of EPO-QD-induced AQPN-4 expression but they further exemplify the minimal impact of the QD scaffold on cellular viability.

### Further Embodiments

This ‘multivalent display’ approach can be applied to other growth factor systems (e.g., platelet-derived growth factor receptor) wherein the simultaneous ligation of multiple receptors at the same time results in enhanced intracellular signaling.

As compared to the QDs used in the examples, other sizes or types of QDs could be used. Furthermore, other types of nanoparticles (gold nanoparticles, liposomes, metal nanoparticles) could be used in a similar fashion while simultaneously taking advantage of their inherent physicochemical properties (e.g., fluorescence, photothermal properties, drug-carrying capacity).

It is expected that the activity and efficacy of the EPO-QD system can be controllably modulated by altering (1) the QD (or other nanoparticle) diameter and/or surface area and/or (2) the separation distance between the QD (or other nanoparticle) surface so as to control the multivalency of interaction between the EPO and the EPOR.

### Advantages

Binding of EPO-QD to astrocytes induces greater induction of expression of AQPN-4 channels compared to free EPO at the same concentration. The increased expression of AQPN-4 by the EPO-QD system translates into an overall increase in both the water efflux rate and magnitude of water efflux in astrocytes compared to free EPO at the same concentration while avoiding impacts on cellular viability.

### CONCLUDING REMARKS

All documents mentioned herein are hereby incorporated by reference for the purpose of disclosing and describing the particular materials and methodologies for which the document was cited.

Although the present invention has been described in connection with preferred embodiments thereof, it will be appreciated by those skilled in the art that additions, deletions, modifications, and substitutions not specifically described may be made without departing from the spirit and scope of the invention. Terminology used herein should not be construed as being “means-plus-function” language unless the term “means” is expressly used in association therewith.

### REFERENCES

1. Jessen, N. et al. (2015) The Glymphatic System: A Beginner's Guide. Neurochem Res. 40:2583-2599
2. Good C. et al. (2020) Sleep in the United States Military. Neuropsychopharmacology. 2020 45: 176-191.
3. Iliff, J. et al (2012) A paravascular pathway facilitates CSF flow through the brain parenchyma and the clearance of interstitial solutes, including amyloid β. Sci Transl Med. 4: 147ra111.
4. Siddiqui, M. et al. (2018) Erythropoietin-mediated activation of aquaporin-4 channel for the treatment of experimental hydrocephalus. Child's Nervous System. 34: 2195-2202.
5. Susumu, K. et al. (2011) Multifunctional compact zwitterionic ligands for preparing robust biocompatible semiconductor quantum dots and gold nanoparticles. J. Am. Chem. Soc. 133: 9480-9486.
6. Kitchen, P. et al. (2020) Calcein fluorescence quenching to measure plasma membrane water flux in live mammalian cells. STAR Protocols 1: 100157.

1. A method of inducing expression of aquaporin-4, the method comprising:
providing a bioconjugate comprising a quantum dot bound to human erythropoietin; and
contacting human astrocytes with the bioconjugate, thereby inducing expression of aquaporin-4 by the human astrocytes.

2. The method of claim 1, wherein the quantum dot comprises
a cadmium selenide core;
an inner cadmium sulfide shell in contact with the cadmium selenide core; and
an outer zinc sulfide shell in contact the inner cadmium sulfide shell.

3. The method of claim 1, wherein the quantum dot is a gold nanoparticle, a non-gold metal nanoparticle, or a liposome.

4. The method of claim 1, wherein the quantum dot comprises a solubilizing ligand.

5. The method of claim 4, wherein the solubilizing ligand is N-(2-carboxyethyl)-N-[2-[(6,8-dimercapto-1-oxooctyl)amino]ethyl]-β-alanine.

6. The method of claim 1, wherein the human erythropoietin comprises a poly-histidine tail.

7. The method of claim 1, wherein the quantum dot has from 2 to 40 molecules of the human erythropoietin deposited thereon.

8. A composition comprising:
a bioconjugate comprising a quantum dot bound to human erythropoietin.

9. The composition of claim 8, wherein the quantum dot comprises:
a cadmium selenide core;
an inner cadmium sulfide shell in contact with the cadmium selenide core; and
an outer zinc sulfide shell in contact the inner cadmium sulfide shell.

10. The composition of claim 8, wherein the quantum dot is a gold nanoparticle, a non-gold metal nanoparticle, or a liposome.

11. The composition of claim 8, wherein the quantum dot comprises a solubilizing ligand.

12. The composition of claim 11, wherein the solubilizing ligand is N-(2-carboxyethyl)-N-[2-[(6,8-dimercapto-1-oxooctyl)amino]ethyl]-β-alanine.

13. The composition of claim 8, wherein the human erythropoietin comprises a poly-histidine tail.

14. The composition of claim 8, wherein the quantum dot has from 2 to 40 molecules of the human erythropoietin deposited thereon.

15. A method comprising:
providing a quantum dot; and
forming a bioconjugate by depositing human erythropoietin onto the surface of the quantum dot.

16. The method of claim 15, wherein the quantum dot comprises:
a cadmium selenide core;
an inner cadmium sulfide shell in contact with the cadmium selenide core; and
an outer zinc sulfide shell in contact the inner cadmium sulfide shell.

17. The method of claim 15, wherein the quantum dot is a gold nanoparticle, a non-gold metal nanoparticle, or a liposome.

18. The method of claim 15, wherein the quantum dot comprises a solubilizing ligand.

19. The method of claim 18, wherein the solubilizing ligand is N-(2-carboxyethyl)-N-[2-[(6,8-dimercapto-1-oxooctyl)amino]ethyl]-β-alanine.

20. The method of claim 15, wherein the human erythropoietin comprises a poly-histidine tail.

21. The method of claim 15, wherein from 2 to 40 molecules of the human erythropoietin are deposited onto the quantum dot.
